SEQUENCE LISTING (6 sequences)

SEQ ID NO: 1 - DNA, 2508 bp, Homo sapiens
caaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatagaattttattgctggaatctactagaaccttctaatccatgtgctgctgtggcatcaggaaaggaagatgtaagaagctaa

SEQ ID NO: 2 - protein, 630 aa, Homo sapiens
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV

SEQ ID NO: 3 - DNA, 3946 bp, Homo sapiens
accgctccggagcgggaggggaggcttcgcggaacgctctcggcgccaggactcgcgtgcaaagcccaggcccgggcggccagaccaagagggaagaagcacagaattcctcaactcccagtgtgcccatgagtaagagcaaatgctccgtgggactcatgtcttccgtggtggccccggctaaggagcccaatgccgtgggcccgaaggaggtggagctcatccttgtcaaggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggcggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctctacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactccctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcagcctgggccgtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcctgcaatgggagagacacgaacaaaccaaggaaatctaagtttcgagagaaaggagggcaacttctactcttcaacctctactgaaaacacaaacaacaaagcagaagactcctctcttctgactgtttacacctttccgtgccgggagcgcacctcgccgtgtcttgtgttgctgtaataacgacgtagatctgtgcagcgaggtccaccccgttgttgtccctgcagggcagaaaaacgtctaacttcatgctgtctgtgtgaggctccctccctccctgctccctgctcccggctctgaggctgccccaggggcactgtgttctcaggcggggatcacgatccttgtagacgcacctgctgagaatccccgtgctcacagtagcttcctagaccatttactttgcccatattaaaaagccaagtgtcctgcttggtttagctgtgcagaaggtgaaatggaggaaaccacaaattcatgcaaagtcctttcccgatgcgtggctcccagcagaggccgtaaattgagcgttcagttgacacattgcacacacagtctgttcagaggcattggaggatgggggtcctggtatgtctcaccaggaaattctgtttatgttcttgcagcagagagaaataaaactccttgaaaccagctcaggctactgccactcaggcagcctgtgggtccttgtggtgtagggaacggcctgagaggagcgtgtcctatccccggacgcatgcagggcccccacaggagcgtgtcctatccccggacgcatgcagggcccccacaggagcatgtcctatccctggacgcatgcagggcccccacaggagcgtgtactaccccagaacgcatgcagggcccccacaggagcgtgtactaccccaggacgcatgcagggcccccactggagcgtgtactaccccaggacgcatgcagggcccccacaggagcgtgtcctatccccggaccggacgcatgcagggcccccacaggagcgtgtactaccccaggacgcatgcagggcccccacaggagcgtgtactaccccaggatgcatgcagggcccccacaggagcgtgtactaccccaggacgcatgcagggcccccatgcaggcagcctgcagaccaacactctgcctggccttgagccgtgacctccaggaagggaccccactggaattttatttctctcaggtgcgtgccacatcaataacaacagtttttatgtttgcgaatggctttttaaaatcatatttacctgtgaatcaaaacaaattcaagaatgcagtatccgcgagcctgcttgctgatattgcagtttttgtttacaagaataattagcaatactgagtgaaggatgttggccaaaagctgctttccatggcacactgccctctgccactgacaggaaagtggatgccatagtttgaattcatgcctcaagtcggtgggcctgcctacgtgctgcccgagggcaggggccgtgcagggccagtcatggctgtcccctgcaagtggacgtgggctccagggactggagtgtaatgctcggtgggagccgtcagcctgtgaactgccaggcagctgcagttagcacagaggatggcttccccattgccttctggggagggacacagaggacggcttccccatcgccttctggccgctgcagtcagcacagagagcggcttccccattgccttctggggagggacacagaggacagtttccccatcgccttctggttgttgaagacagcacagagagcggcttccccatcgccttctggggaggggctccgtgtagcaacccaggtgttgtccgtgtctgttgaccaatctctattcagcatcgtgtgggtccctaagcacaataaaagacatccacaatggaaaaaaaaaaaggaattc

SEQ ID NO: 4 - protein, 620 aa, Homo sapiens
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 5 - DNA, 1983 bp, Homo sapiens
gccggacacagcctcggcgtgcccccaggaccggtaaagttcctctcgccagccgcatccatgcttctggcgcggatgaacccgcaggtgcagcccgagaacaacggggcggacacgggtccagagcagccccttcgggcgcgcaaaactgcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgccttcttgatcccgtacacactgttccttatcatcgcggggatgcccctgttctacatggagctggctctgggacagtacaaccgggagggggctgccaccgtttggaaaatctgcccattcttcaaaggcgttggctatgctgtcatcctgatcgccctgtacgttggcttctactacaacgtcatcatcgcctggtcactctactacctcttctcctccttcaccctcaacctgccctggaccgactgtggccacacctggaacagccccaactgtaccgaccccaagctcctcaatggctccgtgcttggcaaccacaccaagtactccaagtacaagttcacgccggcagccgagttttatgagcgtggtgtcctgcaccttcacgagagcagcgggattcatgacatcggcctgccccagtggcagctcttgctctgtctgatggtcgtcgtcatcgtcttgtattttagcctctggaaaggggtgaagacatcaggaaaggtggtgtggatcacagccacgctgccttacttcgtgctgttcgtgctcctggtccatggcgtcacgctgcccggagcctccaatggcatcaatgcctacctgcacatcgacttctaccgcttgaaagaggccacggtatggattgatgccgcaactcagatatttttttccttgggggctggatttggagtattgattgcatttgccagttacaacaaatttgacaacaactgttacagggatgccctgctgaccagcagcatcaactgtatcaccagcttcgtctctgggttcgccatcttctccatccttggttacatggcccatgaacacaaggtcaacattgaggatgtggccacagaaggagctggcctagtgttcatcctgtatccagaggccatttctaccctgtctggatctacattctgggctgttgtgtttttcgtcatgctcctggcgctgggccttgacagctcaatgggaggcatggaggctgtcatcacgggcctggcagatgacttccaggtcctgaagcgacaccggaaactcttcacatttggcgtcaccttcagcactttccttctcgccctgttctgcataaccaagggtggaatttacgtcttgaccctcctggacacctttgctgcgggcacctccatcctttttgctgtcctcatggaagccatcggagtttcctggttttatggagtggacaggttcagcaacgacatccagcagatgatggggttcaggccgggtctatactggagactgtgctggaagttcgtcagtcctgccttcctcctgttcgtggttgtggtcagcatcatcaacttcaagccactcacctacgacgactacatcttcccgccctgggccaactgggtggggtggggcatcgccctgtcctccatggtcctggtgcccatctacgtcatctataagttcctcagcacgcagggctctctttgggagagactggcctatggcatcacgccagagaacgagcaccacctggtggctcagagggacatcagacagttccagttgcaacactggctggccatctgagcctgcctggaggagaaggaggaacccccatgccaatgtccaggtcacaggcatccgctgctacgtcaa

SEQ ID NO: 6 - protein, 617 aa, Homo sapiens [sequence omitted]

(19) United States
(12) Patent Application Publication
(10) Pub. No.: US 2007/0199084 A1
(43) Pub. Date: Aug. 23, 2007

(54) Ubiquitination of Membrane Transporters

(75) Inventors: Randy D. Blakely, Brentwood, TN (US); Uhna Sung, Nashville, TN (US)

Correspondence Address: FULBRIGHT & JAWORSKI L.L.P., 600 CONGRESS AVE., SUITE 2400, AUSTIN, TX, 78701, US

(73) Assignee: VANDERBILT UNIVERSITY

(21) Appl. No.: 11/552,440

(22) Filed: Oct. 24, 2006

Related U.S. Application Data
(60) Provisional application No. 60/729,720, filed on Oct. 24, 2005.

(51) Int. Cl. A01K 67/027; G01N 33/567

(52) U.S. Cl. 800/18; 435/7.2

(57) ABSTRACT

The present invention addresses methods of screening for ubiquination of membrane-bound transporters, such as SERT, NET and DAT. The ubiquitination of these transporters can regulate their turnover in the cell and trafficking to and from the plasma membrane, and thus provides a novel mechanism to modulate biogenic amine transporter activity.

This application claims benefit of priority to U.S. Provisional Application Serial No. 60/729,720, filed Oct. 24, 2006, the entire contents of which are hereby incorporated by reference.

The government owns rights in the present invention pursuant to grant numbers 2P01HL056693-060001 and R01MH58921 from the National Institutes of Health.

### BACKGROUND OF THE INVENTION

1. Field of the Invention

The present invention relates generally to the fields of neurobiology, pharmacology and psychiatry. More particularly, it concerns the discovery that membrane-bound transporters are ubiquitinated and may thus be regulated by this degradation pathway.

2. Description of Related Art

Neurotransmitters mediate signal transduction in the nervous system and modulate the processing of responses to a variety of sensory and physiological stimuli. An important regulatory step in neurotransmission is the inactivation of a neurotransmitter following its release into the synaptic cleft. This is especially true for the biogenic amine and amino acid neurotransmitters. Inactivation of neurotransmitters is typically mediated by uptake of the released neurotransmitter by neurotransmitters transporters that are located on the presynaptic neuron or in some cases on adjacent glial cells. Thus, neurotransmitter transporters are central to the processing of information in the nervous system and are associated with numerous neurological disorders.

Ubiquitin, a 76-amino acid polypeptide, can be covalently conjugated to lysine residues of target proteins via enzymatic cascades involving E1, E2 and E3 enzymes (Ciechanover and Brundin, 2003). Attachment of ubiquitin to targets proteins for proteolytic degradation through a cellular structure called the proteasome. Degradation of proteins by proteasomes removes denatured, damaged or improperly translated proteins from cells. It also can and regulate the level of proteins such as cyclins, as well as some transcription factors. Enzymes designated as E1 and E2 prepare ubiquitin chains to be attached to proteins by a third enzyme, designated E3. The 20S core proteasome has four rings, each with 14 subunits stacked on top of each other, that are responsible for the proteolytic activity of the proteasome. The PA700 regulatory complex is stacked on the ends of the cylindrical core to form a 26S proteasome. Proteins that have been “tagged” with ubiquitin are recognized and bound by the regulatory subunits, then unfolded in an ATP-dependent manner, and inserted into the core particle, where proteases degrade the protein, releasing small peptides and releasing the ubiquitin intact. Monoubiquitination, as opposed to the polyubiquitination discussed above, has been associated with diverse proteasome-independent cellular functions including intracellular protein movement and plasma trafficking of membrane proteins including ion channels and receptors.

Sung et al. (2004a) first reported that “normal” norepinephrine transporter (NET) appears to be associated with ubiquitin-related enzymes, and that a proteasomal inhibitor triggered accumulation of NET. There was no evidence of NET ubiquitination, however, nor an indication of whether the ubiquitin pathway could regulate NET function. Jiang et al. (2004) showed that mutated/misfold dopamine transporter (DAT) could was ubiquitinated, but did not provide any evidence that “normal” DAT was ubiquitinated, much less regulated by this pathway. Thus, further studies are required to ascertain the true role of the ubiquitin pathway in the modulation of transporter action.

### SUMMARY OF THE INVENTION

Thus, in accordance with the present invention, there is provided a method of screening for agents that affect transporter function comprising (a) providing a membrane-bound transporter; (b) contacting said membrane-bound transporter with a candidate substance; (c) determining the ubiquitination of said transporter; and (d) comparing the ubiquitination of said transporter in step (c) with the ubiquitination of said transporter in the absence of said candidate substance, wherein a candidate substance that alters the ubiquitination of said transporter is an agent that affects transporter function. The transporter may be a norephinephrine transporter (NET), a serotonin transporter (SERT) or a dopamine transporter (DAT). The transporter may be located in an intact cell, such as a neuronal cell or a cell recombinantly engineered to express said transporter. The cell may be from a post-mortem tissue or a tissue biopsy. Alternatively, the transporter may be located in a membrane fragment, or produced by cell-free translation.

The ubiquitination may be determined by an immunoassay (e.g., immunoprecipation, Western blot) or by mass spectrometry. Ubiquitin may be provided exogenously to said cell. The method may further comprise measuring the ubiquitination of said transporter before and after contacting said transporter with said candidate substance. The candidate substance may be a peptide, polypeptide, nucleic acid, lipid, carbohydrate, or organopharmaceutical drug, an enzyme or a nucleic acid encoding an expression construct for an enzyme, such as a protein kinase C, an organopharmaceutical drug that modulates protein kinase C, a ubiquitin-activating enzyme E1A, a ubiquitin substrate, a ubiquitin inhibitor or a ubiquitin hydrolase.

In another embodiment, there is provided a method of modulating neuronal transporter function in a subject comprising administering to said subject a modulator of transporter ubiquitination. The transporter may be a norephinephrine transporter, a serotonin transporter or a dopamine transporter. The subject may be a human, for example, one suffering from mental illness, cardiovascular disease, autonomic dysfunction, ADHD or drug abuse. The modulator may be a peptide, polypeptide, nucleic acid, lipid, carbohydrate, or organopharmaceutical drug, an enzyme or a nucleic acid encoding an expression construct for an enzyme, such as a protein kinase C, an organopharmaceutical drug that modulates protein kinase C, a ubiquitin-activating enzyme E1A, a ubiquitin substrate, a ubiquitin inhibitor or a ubiquitin hydrolase.

In yet another embodiment, there is provided a transgenic mouse encoding a mutant transporter gene, the product of which exhibits reduced or no ubiquitination. The mouse may be homozygous or heterozygous for said mutant transporter gene.

It is contemplated that any method or composition described herein can be implemented with respect to any other method or composition described herein. The use of the word, “a” or “an” when used with the term “comprising” in the specification and/or claims may mean “one,” “one or more,” “at least one,” or “one or more than one.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A—Immunoprecipitation of ubiquitinated NET. cDNAs of NET and HA-Ub were transfected into CAD cells 2 days prior to immunoprecipitation (IP). Left: The complexes were retrieved by IP with anti-HA for Ub, and probed with anti-NET. Right: The complexes were retrieved by IP with anti-NET, and immuno-blotted with anti-HA for Ub. N (NET only), N+U (NET and Ub co-transfection), U (Ub only).

FIG. 1B—Increase of the sizes of NET by ubiquitination. CAD cells were transfected with NET and HA-Ub, immunoprecipitated with anti-HA for Ub, then probed with anti-NET. Note the increase of sizes in immunoprecipitated NET (NET:Ub complexes), compared to NET in total. Right panel: Size of proteins were measured form the left panel of western blot. Standard curve was prepared using migration pattern of molecular weight (MW) marker to determine sizes. Both 90 kDa and 60 kDa bands were shifted to higher MW forms by 7 kDa. Size of high MW bands at the top of gel was not determined.

FIG. 2A—Ubiquitination of DAT (dopamine transporters) and SERT (serotonin transporters) in transfected CAD cells. cDNAs for HA-ubiquitin (Ub) and DAT (Left) or SERT (Right) were cotransfected into CAD cells 2 days prior to immunoprecipitation (IP). The complexes of Ub: transporters were immunoprecipitated by anti-HA and probed with anti-DAT (Left) or anti-SERT (Right). Ubiquitinated DAT and SERT (IP) migrated at larger sizes compared to DAT or SERT in total.

FIG. 2B—Ubiquitination of DAT in striatum. Striatum was dissected from mouse brains in homogenized in 10 mM HEPES, 300 mM sucrose, pH 7.4. Crude synaptosomes were obtained by a sequential centrifugation of a 5 min at 1000×g, followed by a 20 min at 16,000×g. Proteins extracted from the synaptosomes by an one hour incubation in 10 mM HEPES, 300 mM NaCl, 1% TRITON X 100 and protease inhibitors at cold. The lysates were pre-cleared with protein A and G beads. Aliquots of the pre-cleared lysates were incubated with either mouse IgG or monoclonal anti-DAT. Antibodies captured by protein A and G beads were subjected to 10% SDS-PAGE, followed by western blot with anti-Ub. The membranes were stripped and re-probed with anti-DAT.

FIG. 3—Acute regulation of NET ubiquitination. CAD cells were transfected with NET and HA-Ub, and incubated at 37° C. IP was carried out using anti-HA (IP for Ub), followed by immunoblot with anti-NET. Prior to IP, cells were pre-incubated in media containing either vehicle (control), 1 mM PMA, or 1 mM methacholine (meth) for 30 min. PMA and methacholine increased ubiquitination of NET.

FIG. 4—Chronic regulation of NET ubiquitination by anti-depressants. CAD cells were transfected with NET and HA-Ub. After 24 hours, desipramine (Dmi) was added to the cells. CAD cells were incubated for additional 3 days prior to IP. IP was carried out using anti-HA (IP for Ub), followed by immunoblot with anti-NET. Desipramine reduced ubiquitination of NET (IP) and increased NET proteins (total). The “total” membrane were stained with Ponceau-S, showing no change in the amounts of other cellular proteins by desipramine treatment.

FIG. 5A—Inhibition of proteasomes influence NET transport in cells. CAD-his-hNET cells in 24 well plates were incubated in MG132 (10 mM) for 0, 1, 4 and 24 hrs prior to NE transport assay in triplicates.

FIG. 5B—Inhibition of proteasomes influence NET proteins in cells. CAD cells transfected with NET and Ub were preincubated with MG132 for 4 hrs prior to cell lysis and immuno-blot with anti-NET.

### DESCRIPTION OF ILLUSTRATIVE EMBODIMENTS

I. The Present Invention

The norepinephrine transporter (NET) regulates neurotransmission at noradrenergic synapses of the CNS and PNS. Dysfunction of NE clearance or NET density has been associated with attention deficit, mood disorder and suicide (Delgado and Moreno, 2000; Arnsten, 2001; Zaro et al., 2003; Rehman and Masson, 2001; Klimek et al., 1997). Stimulation of presynaptic receptors by drugs or endogenous hormones rapidly regulates the activity of NET by a variety of signaling pathways. The changes in NET activity involve alteration of catalytic activity, surface trafficking, phosphorylation, turnover of NET proteins and interactions with other cellular proteins (Sung et al., 2003; Bauman et al., 2000; Apparsundaram et al., 1998a; Apparsundaram et al., 1998b; Apparsundaram et al., 2001; Jayanthi et al., 2004). Understanding regulatory mechanisms supporting NET activity and NE signaling, and that of other membrane-bound transporters like DAT and SERT, is strategically important for elucidating compromised pathways in psychiatric disorders and to identify novel therapeutic targets.

Ubiquitin, a 76-amino acid polypeptide, can be covalently conjugated to lysine residues of target proteins via enzymatic cascades involving E1, E2 and E3 enzymes (Ciechanover and Brundin, 2003). Ubiquitination of proteins and degradation by the proteasome system plays important roles in synaptic plasticity by controlling stability, activity and localization of target proteins (Ehlers, 2003; Pak and Sheng, 2003; Myat et al., 2002). Ubiquitination modulates surface expression of glutamate receptors and stability of PSD-95, β2-adrenergic receptor and epithelial Na⁺ channels (ENaC) (Burbea et al., 2002; Colledge et al., 2003; Shenoy et al., 2001; Staub et al., 1997; Abriel et al., 1999). Ubiquitination systems have been implicated in a number of neurological diseases such as Parkinson's (PD), Alzheimer's diseases (AD), Lewy body dementia, Huntington's (HD), prion diseases, amyotrophic lateral sclerosis (ALS), motor dysfunction and even mental retardation (Ciechanover and Brundin, 2003; Jiang et al., 1998).

NET has been shown to interact with a number of cellular proteins such as syntaxin 1A, PP2A, PICK1 and Hic-5 (Sung et al., 2003; Bauman et al., 2000; Torres et al., 2001; Carneiro et al., 2002). It is likely that these interactions influence NET activity, trafficking and responses to drugs, although defining interactions and understanding functional importance remain an active area of investigation. In a previous study using co-immunoprecipitation of NET from neuroblastoma cell, as well as MS analysis of NET complexes, the inventors have discovered that ubiquitin (Ub) system enzymes including Nedd-4 (E3 ligase), E1 Ub activating and E2 Ub conjugating enzymes appear to interact with NET. However, definitive evidence of interaction with ubiquitin, and more significantly, evidence of NET ubiquitination, was lacking. The inventors have now determined that NET can be ubiquitinated in a manner sensitive to cellular signaling pathways. Additionally, both DAT and SERT have now been shown to be ubiquitinated.

With the reports that unbalanced NE and region specific alteration of NET density in brain have been associated with major depression and suicide (Klimek et al., 1997; Delgado, 2000; Gross-Isseroff et al., 1989), the identification of Ub and Ub system enzymes in NET complexes, as well as other transporters, suggests that altered ubiquitination of membrane-bound transporters may regulate transporter function and thus contribute to neurologic diseases and the therapeutic action of antidepressants that action through such transporters. Thus, a variety of methods are provided herein to examine ubiquitination of transporters, to screen for compounds that alter their ubiquitination, and to modulate ubiquitination in such a way as to altering transporter function.

II. Membrane-bound Transporters

A. Serotonin Transporter

Serotonin (5-hydroxytryptamine, 5-HT) is a neurotransmitter in the brain and periphery, and modulates a wide variety of physiological processes including vasoconstriction, gastrointestinal motility and secretion, respiration, sleep, appetite, aggression, and mood (Jacobs and Azmitia, 1992; Fozzard, 1989). Disrupted 5-HT signaling has been implicated in a similarly wide-spectrum of disorders including primary pulmonary hypertension, irritable bowel syndrome, sudden infant death syndrome (SIDS), anorexia, obsessive-compulsive disorder (OCD), autism, depression and suicide (Insel et al., 1990; Melzter, 1990; Gershon, 1999; Cook and Leventhal, 1996). A major determinant of 5-HT signaling is the antidepressant-sensitive 5-HT transporter (SERT, 5HTT). Human SERT (hSERT) protein is encoded by a single locus mapping to chromosome 17q11.2 (Ramamoorthy et al., 1993). Although evidence of alternative splicing of 5′ non-coding exons exists (Bradley and Blakely, 1997; Ozsarac et al., 2002), the same open reading frame is translated in brain, platelets, lymphocytes and placenta, producing a protein of 630 amino acids with closest identify to norepinephrine and dopamine transporters (NET and DAT respectively). Initial hydropathy-based predictions of SERT secondary structure proposed 12 transmembrane domains (TMs) with intracellular NH2 and COOH termini (Hoffman et al., 1991), a model supported by biochemical and immunocytochemical studies (Chen et al., 1998; Miner et al., 2000).

B. Dopamine Transporter

The dopamine transporter (DAT) mediates uptake of dopamine into neurons and is a major target for various pharmacologically active drugs and environmental toxins. Since its cloning, much information has been obtained regarding its structure and function. The cloning data predicts that the DAT is a 619 (rat) or 620 (human) amino acid protein. Hydropathy analysis suggests that the DAT includes 12 transmembrane domains (TMDs), with both the amino- and carboxy-termini residing within the cytoplasm, consistent with recent immunochemical data. Monoamine transporter sequences are least conserved at these termini and a large extracellular loop occurring between TMD 3 and TMD 4 and most conserved in the putative TMDs.

Binding domains for dopamine and various blocking drugs including cocaine are likely formed by interactions with multiple amino acid residues, some of which are separate in the primary structure but lie close together in the still unknown tertiary structure. Chimera and site-directed mutagenesis studies suggest the involvement of both overlapping and separate domains in the interaction with substrates and blockers, whereas recent findings with sulfhydryl reagents selectively targeting cysteine residues support a role for conformational changes in the binding of blockers such as cocaine. The dopamine transporter can also operate in reverse, i.e., in an efflux mode, and recent mutagenesis experiments show different structural requirements for inward and outward transport.

C. Norepinephrine Transporter

The norepinephrine transporter (NET) is an antidepressant-sensitive transporter located on plasma membranes of noradrenergic neurons and other specialized cells that remove norepinephrine (NE) from the synapse to terminate the actions of NE. It contains of 617 amino acids and has 12 transmembrane domains. This conformation is similar to that of other membrane-associated proteins that are responsible for ion and solute transport. Missense polymorphisms have been identified in the human NET gene, including the replacement of an alanine residue with a proline residue at position 457 (A457P) that is associated with orthostatic intolerance, the F528C polymorphism, and a R121Q change that has not previously been reported.

D. Protein Compositions

The term “amino acid composition” encompasses amino acid sequences comprising at least one of the 20 common amino acids in naturally synthesized proteins, or at least one modified or unusual amino acid. It is also well understood that where certain residues are shown to be particularly important to the biological or structural properties of a protein, polypeptide or peptide, e.g., residues in binding regions or active sites, such residues may not generally be exchanged. In this manner, functional equivalents are defined herein as those peptides which maintain a substantial amount of their native biological activity. For comparison purposes, the wild-type sequence for human SERT is set forth in SEQ ID NO:1, the wild-type sequence for human DAT is set forth in SEQ ID NO:3, and the wild-type sequence for human NET is set forth in SEQ ID NO:5.

III. Nucleic Acid Molecules

A. Nucleic Acids Encoding Transporters

The present invention also provides membrane-bound transporter-encoding nucleic acids. Nucleic acids of the present invention may be derived from genomic DNA, complementary DNA (cDNA). More particularly, the present invention provides synthetic nucleic acid sequences comprising the amino acid sequences of the human serotonin transporter. Nucleic acids of the present invention also concern isolated DNA segments encoding wild-type, polymorphic or mutant serotonin transporter proteins, polypeptides or peptides. The wild-type human SERT sequence is provided as SEQ ID NO:2, the wild-type human DAT sequence is provided as SEQ ID NO:4, and the wild-type human NET sequence is provided as SEQ ID NO:6.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 20, of about 50 to about 90, of about 100 to about 200, of about 210 to about 300, of about 310 to about 350, of about 360, to about 400, of about 410 to about 450, of about 460 to about 500, of about 510 to about 550, of about 560 to about 600, of about 610 to about 650, of about 660 to about 700, of about 710 to about 750, of about 760 to about 800, of about 810 to about 850, of about 860 to about 900, about 1000, about 1100, about 1200, about 1300, about 1400, about 1500, about 1600, about 1700, about 1800, about 1900 or greater nucleotide residues in length. Those of skill will recognize that in cases where the nucleic acid region encodes a transporter peptide, polypeptide or protein, the nucleic acid region can be quite long, depending upon the number of amino acids in the transporter molecule.

It is contemplated that the transporter may be encoded by any nucleic acid sequence that encodes the appropriate amino acid sequence. The design and production of nucleic acids encoding a desired amino acid sequence is well known to those of skill in the art, using standardized codon tables (Table 1). In preferred embodiments, the codons selected for encoding each amino acid may be modified to optimize expression of the nucleic acid in the host cell of interest. The term “functionally equivalent codon” is used herein to refer to codons that encode the same amino acid, such as the six codons for arginine or serine, and also refers to codons that encode biologically equivalent amino acids. Codon preferences for various species of host cell are well known in the art. Codons preferred for use in humans, are well known to those of skill in the art (Wada et al., 1990). Codon preferences for other organisms also are well known to those of skill in the art (Wada et al., 1990, included herein in its entirety by reference) and can be found on the internet at the Codon Usage Database website.

TABLE 1

| Amino Acid | | | Codons | | | | |
|---|---|---|---|---|---|---|---|
| Alanine | Ala | A | GCA | GCC | GCG | GCU | |
| Cysteine | Cys | C | UGC | UGU | | | |
| Aspartic acid | Asp | D | GAC | GAU | | | |
| Glutamic acid | Glu | E | GAA | GAG | | | |
| Phenylalanine | Phe | F | UUC | UUU | | | |
| Glycine | Gly | G | GGA | GGC | GGG | GGU | |
| Histidine | His | H | CAC | CAU | | | |
| Isoleucine | Ile | I | AUA | AUC | AUU | | |
| Lysine | Lys | K | AAA | AAG | | | |
| Leucine | Leu | L | UUA | UUG | CUA | CUC | CUG |
| | | | CUU | | | | |
| Methionine | Met | M | AUG | | | | |
| Asparagine | Asn | N | AAC | AAU | | | |
| Proline | Pro | P | CCA | CCC | CCG | CCU | |
| Glutamine | Gln | Q | CAA | CAG | | | |
| Arginine | Arg | R | AGA | AGG | CGA | CGC | CGG |
| | | | CGU | | | | |
| Serine | Ser | S | AGC | AGU | UCA | UCC | UCG |
| | | | UCU | | | | |
| Threonine | Thr | T | ACA | ACC | ACG | ACU | |
| Valine | Val | V | GUA | GUC | GUG | GUU | |
| Tryptophan | Trp | W | UGG | | | | |
| Tyrosine | Tyr | Y | UAC | UAU | | | |

In certain embodiments, the invention concerns isolated DNA segments and recombinant vectors that include within their sequence a contiguous nucleic acid sequence encoding the amino acid sequences shown in SEQ ID NO: 2, 4 and 6. As used herein, the term “DNA segment” refers to a DNA molecule that has been isolated free of total genomic DNA of a particular species. Therefore, a DNA segment encoding serotonin transporter refers to a DNA segment that contains wild-type, polymorphic or mutant serotonin transporter coding sequences yet is isolated away from, or purified free from, total mammalian genomic DNA. Included within the term “DNA segment,” are DNA segments and smaller fragments of such segments, and also recombinant vectors, including, for example, plasmids, cosmids, phage, viruses, and the like.

Similarly, a DNA segment comprising an isolated or purified transporter gene refers to a DNA segment for SERT, DAT or NET protein, polypeptide or peptide coding sequences and, in certain aspects, regulatory sequences, isolated substantially away from other naturally-occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and engineered segments that express, or may be adapted to express, proteins, polypeptides, domains, peptides, fusion proteins and mutants of transporter encoded sequences.

“Isolated substantially away from other coding sequences” means that the gene of interest, in this case the serotonin, dopamine or norepinephrine transporter gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments that encode a transporter protein, polypeptide or peptide that includes within its amino acid sequence a contiguous amino acid sequence in accordance with, or essentially as set forth in, SEQ ID NO: 2, 4 or 6. The term “a sequence essentially as set forth in SEQ ID NO: 2, 4 or 6” means that the sequence substantially corresponds to a portion of SEQ ID NO: 2, 4 or 6 and has relatively few bases that are not identical to, or a biologically functional equivalent of (i.e., encode amino acid), SEQ ID NO: 2, 4 or 6.

It will also be understood that nucleic acid sequences may include additional residues, such as additional 5′ or 3′ sequences, and yet still be essentially as set forth in one of the sequences disclosed herein. The addition of terminal sequences particularly applies to various non-coding sequences flanking either of the 5′ or 3′ portions of the coding region or may include various internal sequences, i.e., introns, which are known to occur within genes.

Excepting intronic or flanking regions, and allowing for the degeneracy of the genetic code, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of nucleotides that are identical to the nucleotides of SEQ ID NO: 2, 4 or 6.

In addition to the “standard” DNA and RNA nucleotide bases, modified bases are also contemplated for use in particular applications of the present invention. A table of exemplary, but not limiting, modified bases is provided herein (Table 2).

TABLE 2
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | -alanine, -Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In addition to nucleic acids encoding a transporter, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture. Such sequences include probes for assessing transporter expression and structure, and primers for amplifying and/or sequencing transporter genes.

B. Vectors

The present invention contemplates the transfer or wild-type and variant transporter molecules into host cells. Virtually any type of vector may be employed in any known or later discovered method to deliver nucleic acids encoding a transporter. Such vectors may be viral or non-viral vectors as described herein, and as known to those skilled in the art. U.S. Pat. Nos. 5,312,734, 5,418,162, and 5,424,185, all incorporated herein by reference, describe nucleic acids, vectors, and host cells used to express various neurotransmitter transporters in cells.

1. Expression Constructs

A vector in the context of the present invention refers to a carrier nucleic acid molecule into which a nucleic acid sequence encoding a serotonin transporter can be inserted for introduction into a cell and thereby replicated. A nucleic acid sequence can be exogenous, in that it is foreign to the cell into which the vector is being introduced; or that the sequence is homologous to a sequence in the cell but positioned within the host cell nucleic acid in which the sequence is ordinarily not found. One of skill in the art would be well equipped to construct a vector through standard recombinant techniques as described in Sambrook et al., 2001; Maniatis et al., 1990; and Ausubel et al., 1994 (each incorporated herein by reference).

It is contemplated in the present invention, that virtually any type of vector may be employed in any known or later discovered method to deliver nucleic acids encoding a transporter peptide, polypeptide or protein, or constructs thereof. Such vectors may be viral or non-viral vectors as described herein, and as known to those skilled in the art.

An expression vector of the present invention refers to any type of genetic construct comprising a nucleic acid coding for a RNA capable of being transcribed. In some cases, RNA molecules are translated into a protein, polypeptide, or peptide. An expression construct comprising a nucleic acid encoding a transporter peptide, polypeptide, or protein may comprise a virus or engineered construct derived from a viral genome and may also comprise a natural intron or an intron derived from another gene. In other cases, these sequences are not translated as in the case of antisense molecules or ribozymes production. Expression vectors can contain a variety of control sequences, which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well, and are described herein. Additionally, as set forth above one may also use mutant versions, isoforms, and other variants of any transporter in the methods of the invention. The foregoing section provides a general description of how exogenous expression may be achieved.

Expression requires that appropriate signals be provided in the vectors, which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

a. Promoters and Enhancers

Expression requires that appropriate signals be provided in the vectors, and which include various regulatory elements, such as enhancers/promoters from both viral and mammalian sources that drive expression of the genes of interest in host cells. Throughout this application, the term “expression construct” is meant to include any type of genetic construct containing a nucleic acid coding for a gene product in which part or all of the nucleic acid encoding sequence is capable of being transcribed and translated into a polypeptide product. An “expression cassette” is defined as a nucleic acid encoding a gene product under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

By attaching a tissue-specific or cell-specific promoter region of a nucleic acid to a reporter or a detectable marker, one can obtain tissue-specific or cell-specific expression. The present invention particularly contemplates the use of the serotonin promoter to drive expression of the nucleic acid of interest.

By employing a promoter with well-known properties, the level and pattern of expression of the protein of interest following transfection or transformation can be optimized. Further, selection of a promoter that is regulated in response to specific physiologic signals can permit inducible expression of the gene product. Any promoter/enhancer combination (as per the Eukaryotic Promoter Data Base EPDB) may be used to drive expression of the gene. Eukaryotic cells can support cytoplasmic transcription from certain bacterial promoters if the appropriate bacterial polymerase is provided, either as part of the delivery complex or as an additional genetic expression construct.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

C. Polyadenylation and Termination Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells.

Where a cDNA is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals.

Also contemplated as an element of the expression cassette is a transcriptional termination site. The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

These elements can serve to enhance message levels and/or to minimize read through from the cassette into other sequences.

d. Splicing Sites and Origins of Replication

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. See Chandler et al. (1997), incorporated herein by reference).

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

e. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999; Levenson et al., 1998; and Cocea, 1997; incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

2. Selectable Markers

In certain embodiments of the invention, the cells contain nucleic acid constructs encoding a transporter may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (see the atcc website on the internet). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5, JM109, and KC8, as well as a number of commercially available bacterial hosts such as SURE® Competent Cells and SOLOPACK Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris. Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Of particular interest are neuronal cell lines and primary cultures.

D. Viral Transfer

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubinstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubinstein, 1988; Temin, 1986).

The retroviruses are a group of single-stranded RNA viruses characterized by an ability to convert their RNA to double-stranded DNA in infected cells; they can also be used as vectors. Adenoviruses are also typically used as vectors due to their mid-sized genome, ease of manipulation, high titer, wide target cell range and high infectivity. The use of retroviral and adenoviral vectors in eukaryotic gene expression and gene therapy are well known in the art. Other viral vectors may also be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. These vectors offer several attractive features for various mammalian cells (Friedmann, 1989; Ridgeway, 1988; Baichwal and Sugden, 1986; Coupar et al., 1988; Horwich et al., 1990).

E. Non-Viral Transfer of Nucleic Acids Encoding Transporters

There are a number of suitable methods by which nucleic acids encoding amino acid sequences of the transporter may be introduced or delivered to cells. Virtually any method by which nucleic acids (e.g., DNA, including viral and nonviral vectors) can be introduced into a cell, or an organism may be employed with the current invention, as described herein or as would be known to one of ordinary skill in the art. Several methods for the transfer of expression constructs into mammalian cells include, but are not limited to: direct delivery of DNA by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods.

IV. Screening Methods

A. Screening for Modulators of Transporter Ubiquination

Defects in transporter are associated with various nervous system disorders including depression, stress disorders, attention deficit disorder, anxiety, obesity, several sleep related disorders and certain neurodegenerative diseases (Edwards, 1993). As described herein, the inventors have now demonstrated that the ubiquitin pathway can affect transporter activity, and further, that drugs affecting transporter function can alter ubiquitination.

The present invention therefore provides methods for screening of drugs that affect ubiquitination of transporters, and hence transporter function. The drugs may be known therapeutics, or may be members of large libraries of candidate substances. The activities examined may include binding to receptors, uptake by receptors, accumulation in cells, or clearance of the neurotransmitter, its analog or derivative. Micro-dialysis and amperometry may be used to assay transporter function in vivo (Giros et al., 1996; Galli et al., 1998).

Assays may be conducted in cell free systems such as cellular extracts, cell membrane preparations which may be prepared by lysing cells, in isolated cells, in cells that express endogenous transporter, in cells that are genetically engineered to express the transporter, in cells that exogenously or endogenously express altered, mutant or functionally-deficient transporters, or in organisms including transgenic animals or animal models of diseases wherein the disease is associated with neurotransmitter transporters.

It will, of course, be understood that all the screening methods of the present invention are useful in themselves notwithstanding the fact that effective candidates may not be found. The invention provides methods for screening for such candidates, not solely methods of finding them.

B. Assessing Ubiquitination

There are a variety of methods for examining whether a protein is ubiquitinated, and to what extent. In one assay, transporters will be immunoprecipitated using an anti-transporter antibody, and the ubiquitination of the transporter will be assessed following separation (e.g., electrophoresis) by a label on the ubiquitin or by a shift in the molecular weight of the transporter. A general protocol involves growth of cells in tissue culture plates, followed by harvesting by scraping in PBS/0.5 mM PMSF. Harvested cells are extracted with PBS/1% Triton X 100/0.5 mM PMSF/protease inhibitors at 4° C. for 1 hour to overnight. Cell lysates are recovered by centrifugation and incubated with mouse IgG coupled sepharose beads for 1 hour to reduce nonspecific binding proteins. Anti-HA, coupled to agarose beads, are then added to the pre-cleared extracts, incubated at 4° C. overnight, washed with PBS/1% Triton X 100/0.5 mM PMSF. Bound proteins are eluted in 0.1 M glycine (pH 2.0). Aliquots of eluted proteins are analyzed by 10% SDS-PAGE, probed for transporter, or visualized by silver staining. A variation on this approach is to omit the labeled ubiquitin, and rather, to detect ubiquitin by the use of an antibody that binds selectively to a ubiquitinated form of a protein.

An alternative approach is to use mass spectrometric analysis. Mass spectrometry (MS) is a powerful method for detecting variations in proteins. By exploiting the intrinsic properties of mass and charge, mass spectrometry (MS) can resolve and confidently identify a wide variety of complex proteinaceous compounds. Mass spectrometry is an excellent tool for identifying histones and histone modifications.

There are a variety of mass spectrometry techniques known in the art. Traditional quantitative MS has used electrospray ionization (ESI) followed by tandem MS (MS/MS) (Chen et al., 2001; Zhong et al., 2001; Wu et al., 2000). Of particular interest in the present invention is matrix assisted laser desorption/ionization time of flight (MALDI-TOF) MS (Bucknall et al., 2002; Mirgorodskaya et al., 2000; Gobom et al., 2000). Another MS technique of interest in the present invention is surface enhanced laser desorption/ionization time of flight (SELDI-TOF) MS, which is a variation on MALDI-TOF. While MALDI-TOF and SELDI-TOF are preferred MS techniques, the present invention is not limited to a particular type of MS.

1. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; 6,756,586, 5,572,023 and 5,986,258.

2. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000). Desorption electrospray is a new associated technique for sample surface analysis.

3. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles.

4. LD-MS and LDLPMS

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation require a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

5. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Zaluzec et al., 1995; Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

Identification of proteins corresponding to predictive MALDI-TOF signals typically involves two approaches. First, protein extracts from tissue samples will be fractionated by HPLC, 1D SDS-PAGE or solution phase isoelectric focusing and fractions exhibiting the MALDI-TOF MS signals of interest will be subjected to tryptic digestion and analysis by LC-MS-MS. Peptides and their corresponding proteins of origin are identified from MS-MS spectra with Sequest, which correlates uninterpreted MS-MS spectra with theoretical spectra from database sequences (Eng et al., 1994). Confirmation of the protein identities is based on apparent molecular weight of the MS-MS identified proteins compared to pattern-specific signals detected in the MALDI profiles.

A second identification approach will pair LC-MS-MS analyses with stable isotope tags. Protein extracts from two samples to be compared (e.g., samples that differ in MALDI proteome patterns) are chemically tagged with light and heavy (unlabeled vs. deuterium or ¹³C-labeled) reagents, then combined, digested and the tagged peptides are then analyzed by LC-MS-MS. Peptides derived from the two samples are distinguished by pairs of signals in full scan MS separated by the mass difference of the light and heavy isotope tags. Pairs of signals whose intensities deviate from unity represent proteins that were differentially present in the original two samples. MS-MS spectra acquired from these peaks in the same LC-MS-MS analyses allow unambiguous identification of the differentially expressed proteins. The best-known version of this approach uses the thiol-reactive ICAT reagents developed by Gygi and Aebersold (Gygi et al., 1999), although newer, acid-cleavable reagents offer more efficient recovery of tagged peptides and produce higher quality MS-MS spectra for identification (Zhou et al., 2002). N-terminal isotope tagging of tryptic peptides enables identification of proteins that differ in posttranslational modifications rather that protein expression level per se (Mason and Liebler, 2003).

6. SELDI-TOF MS

Surface-enhanced laser desorption ionization-time of flight mass spectrometry (SELDI-TOF MS) is a variant of MALDI-TOF mass spectrometry. In SELDI-TOF MS, fractionation based on protein affinity properties is used to reduce sample complexity. For example, hydrophobic, hydrophilic, anion exchange, cation exchange, and immobilized-metal affinity surfaces can be used to fractionate a sample. The proteins that selectively bind to a surface are then irradiated with a laser. The laser desorbs the adherent proteins, causing them to be launched as ions. The “time of flight” of the ion before detection by an electrode is a measure of the mass-to-charge ration (m/z) of the ion.

C. In vitro Assays

In particular embodiments, the present invention provides a method for screening transporters in an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay in this invention is the use of cellular extracts that comprise a ubiquitin pathway enzymes, and optionally ubiquitin. These may be cell membrane preparations that comprise a transporter, particularly SERT, DAT or NET. While not directly addressing transpoter function, the ability of candidate substance to alter transporter complexing with ubiquitin pathway enzymes is strong evidence of a related biological effect. Alternatively, a cell-free ubiquitination assay can be employed. Usually, the target transporter will be labeled with ubiqutin.

D. In cyto Assays

Various cells and cell lines can also be utilized for screening assays for drug effects on transporter ubiquitination. This includes cells specifically engineered to express or overexpress a transporter. Such cells and nucleic acid vectors are described in several sections infra. Cells contemplated in the present invention include, but are not limited to, neuronal cells. Such neuronal cells may include post-mortem- or biopsy-obtained primary cells. Depending on the assay, culture may be required. The cell is examined using any of a number of different physiologic assays designed at assessing transporter function. Alternatively, the analysis may simply be one to assess transporter ubiquitination or degradation.

1. Measurement of Transport

In some embodiments, the present invention will examine transport of a neurotransmitter by a transporter that comprises the measurement of uptake and/or accumulation of neurotransmitter and analogues thereof that are specifically taken up by the transporter. Typically, this is accomplished by measuring the uptake or binding of radiolabeled neurotransmitter, e.g., serotonin, or a radiolabeled antagonist, e.g., citalopram, paroxetine, or RTI-55. Conventional assays involves the uptake of radiolabeled 5HT where antagonist sensitivity is measured for inhibition of serotonin accumulation or the inhibition of labeled antagonist binding to intact cells expressing SERT or to membranes from intact cells expressing SERT. Basically, cells transfected with a SERT construct are washed in assay buffer followed by a preincubation in 37° C. assay buffer containing 1.8 g/L glucose. This is followed by an incubation period, about 10 minutes, at 37° C. in the presence of [³H]-5-HT, or a radiolabeled antagonist.

Vaughan et al. (1991) describe a dopamine transporter assay using the cocaine analog [³H]WIN 35,428 for labeling of digitonin-solubilized dopamine transporters from dog caudate nucleus. The assay involves incubation of extracts with the ligand followed by separation of free from bound ligand by centrifugation after adding activated charcoal. Specific binding was observed in dog caudate but was absent in dog cerebellar extracts. Binding was linear with tissue, saturable, and of high affinity (Kd=16 nM). In competition studies, soluble [³H]WIN 35,428 binding was inhibited strongly by mazindol, GBR 12909, and (−)-cocaine but only weakly by citalopram, desipramine, and (+)-cocaine; this is typical of binding to the dopamine transporter. Compared to assays using [³H]GBR 12935, (−)-cocaine was relatively more potent, suggesting that the cocaine and GBR 12935 binding sites are somewhat different. When soluble extract was chromatographed on a wheat germ agglutinin-Sepharose column, [³H]WIN 35,428 binding activity was eluted with N-acetylglucosamine in a manner similar to photoaffinity-labeled dopamine.

Norepinephrine transporter assays may be conducted as described by Kocabas et al. (2003). Briefly, at 16 h post-transfection, cells were washed and incubated for 10 min with phosphate-buffered saline (PBS) containing 0.1 mM CaCl₂ and 1 mM MgCl₂ (PBS/CM). Transport functions were measured by incubating the cells with either 20.5 nM [³H]serotonin (3500 cpm/pmol; NEN Life Science Products, Boston, Mass., U.S.A.) or 28.7 nM[³H]dopamine (DA; 1619 cpm/pmol; NEN Life Science Products) in PBS/CM for 10 min at room temperature, an interval previously determined to include only the initial linear phase of transport. The intact cells were quickly washed with ice-cold PBS, lyzed in sodium dodecyl sulfate (SDS), transferred to scintillation vials and counted as described previously (Blakely et al., 1991; Kilic & Rudnick, 2000). The effects of MTSET or citalopram were tested by including the inhibitors in the 250 μL of PBS/CM during a 10-min pre-incubation step before addition of substrate. Results are from triplicate samples and were repeated in two to three separate experiments.

Other transporter assays are described below.

a. Scintillation Proximity Assays

Measurement of transport may also be involve scintillation proximity assays, which is used to count the accumulated radiolabel on plates having scintillant embedded in them. Basically, cells are plated at 50% confluence on 0.4-μm pore size 6.5-mm Transwell cell culture filter inserts and grown for 7 days. A cell monolayer growing on the porous membrane of the cell culture filter insert effectively separates each well in the cell culture plate into two chambers. The apical membranes of epithelial cells plated on these filters faces the chamber above the cells and the basolateral membranes face the lower chamber through the filter. After one wash each of the apical (upper chamber) and basolateral (lower chamber) sides of the monolayer with PBS/Ca/Mg, the cells are incubated in PBS/Ca/Mg containing ³H-labeled substrate either in the upper or the lower chamber at 22° C. At the end of the incubation cells are washed either three times from the apical side and once from the basolateral side (when ³H-labeled substrate was present in the upper chamber) or once from the apical side and three times from the basolateral side (when substrate was present in the lower chamber). The apical side of the cells are washed by adding 0.2 ml of ice-cold PBS to the upper chamber and aspirating. The basolateral side of the cells are washed by pipetting ice-cold PBS over the bottoms of the filter inserts. After the washes, the filters with cells attached are excised from the insert cups, submerged in 3 ml of Optifluor scintillation fluid (Packard Instrument Co., Downers Grove, Ill.), and counted in a Beckman LS-3801 liquid scintillation counter. Transport assays on 48-well plates were described previously (Gu et al., 1994).

b. Voltage and Patch Clamp

The present invention also employs a means of determining transporter activity or function by measuring the change in movement across a membrane, when the transporter is active. This may be accomplished using the voltage clamp technique, as is well known in the art, this allows the gating properties of the voltage-gated channels to be analyzed.

In short, the voltage clamp technique is a procedure whereby the transmembrane voltage of a membrane segment is rapidly set and maintained at a desired level. Once the membrane potential is controlled, the current flowing through the channels in that segment can be measured.

The patch clamp technique allows the voltage clamp technique to be applied to a small patch of membrane containing a single voltage-sensitive channel. The basic idea behind a patch clamp experiment is to isolate a patch of membrane so small that it contains a single voltage-gated channel. Once this patch of membrane is isolated, the single channel can be voltage clamped. Using this technique, the gating properties of the serotonin transporter can be characterized.

2. Other Methods of Measurement of Transport

Other methods of measurement contemplated in the present invention may involve fluorescence microscopy. This may involve the use of fluorescent substrates, some of which are contemplated to be analogs of other native. neurotransmitters.

a. Microscopy

Fluorescent microscopy is used to measure transport using neurotransmitters or analogues thereof which are fluorescent substrates for the transporter. Cells that either endogenously or exogenously express a transporter are isolated and plated on glass bottom Petri-dishes or multi-well plates that may typically be coated with poly-L-lysine or any other cell adhesive agent. Cells are typically cultured for three or more days. The culture medium is then aspirated and the cells are mounted on a Zeiss 410 confocal microscope. During the confocal measurement cells remain without buffer for approximately thirty seconds. Background autofluorescence is established by collecting images for ten seconds prior to the addition of the buffer and neurotransmitter or analogues thereof. As the neurotransmitter or an analogue thereof has a large Stoke shift between excitation (lₘₐₓ=488 nm) and emission maxima (lₘₐₓ=610 nm), the argon laser is tuned to 488 nm and the emitted light filtered with a 580-630 nm band pass filter (lₘₐₓ=610 nm). The substantial red shift can be exploited to reduce background auto-fluorescence produced in the absence of substrate. The gain (contrast) and offset (brightness) for the photomultiplier tube (PMT) may be set to avoid detector saturation at the higher neurotransmitter concentrations that may be used in certain experiments. The effects of photo-bleaching on neurotransmitter accumulation may also be determined by examining the rate of neurotransmitter accumulation and decay at various acquisition rates.

b. Fluorescence Anisotropy Measurements

To evaluate neurotransmitter binding to the surface membranes, cells expressing a transporter may be exposed to the neurotransmitter or analogues thereof with horizontal polarizer, with the polarizer rapidly switching to the vertical position. Cells may be imaged with alternating polarizations for 3 minutes to measure light intensity in the horizontal (Iₕ) and vertical (Iᵥ) positions in order to calculate the anisotropy ratio, r=(Iᵥ−gIₕ)/(Iᵥ+2 g Iₕ). The factor g may be determined by using a half wave plate as described by Blackman et al. (1996). In this formulation, r=0.4 implies an immobile light source. Surface anisotropy can be measured at the cell circumference over 1 pixel width (0.625 mm). Cytosolic anisotropy can be measured near the center of the cell, approximately 5 pixel widths from the membrane.

C. Image Analysis

The fluorescent images may be processed using suitable software. For example, fluorescent images may be processed using MetaMorph imaging software (Universal Imaging Corporation, Downington Pa.). Fluorescent accumulation may be established by measuring the average pixel intensity of time resolved fluorescent images within a specified region identified by the DIC image. Average pixel intensity is used to normalize among cells.

d. Single Cell Fluorescence Microscopy

In some embodiments, the invention provides measurement of transporter characteristics at the single-cell level. Single-cell fluorescence microscopy provides a powerful assay to study rapid transmitter uptake kinetics from single cells.

e. Automation

The inventors further contemplate that all these methods are adaptable to high-throughput formats using robotic fluid dispensers, multi-well formats and fluorescent plate readers for the identification of transporter modulators.

E. In vivo Assays

In vivo assays are also contemplated in the present invention for secondary screening of drugs for effects on transporter function and/or ubiquitination. Such assays involve the use of various animal models, including transgenic animals that have been engineered to have specific defects, or carry markers that can be used to measure the ability of a candidate substance to reach and effect activity and/or ubiquination of transporters in different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors or blockers of the serotonin transporter may be conducted using an animal model derived from any of these species.

In such assays, one or more candidate substance is administered to an animal, and the ability of the candidate substance(s) to alter one or more characteristics that are a result of transporter function or activity and/or ubiquitination, as compared to a similar animal not treated with the candidate substance(s), identifies a modulator. The characteristics may be any of those discussed above with regard to the function or activity of the transporter, such as change in neurotransmission, change in the activity of some other downstream protein due to a change in neurotransmission, or instead a broader indication such as behavior of an animal, etc.

Treatment of animals with candidate substance(s) will involve the administration of the substance, in an appropriate form, to the animal. Administration will be by any route that could be utilized for clinical or non-clinical purposes, including but not limited to oral, nasal, buccal, or even topical. Alternatively, administration may be by parenteral methods such as intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

1. In vivo Microdialysis

Microdialysis may be used in the present invention to monitor interstitial fluid in various body organs with respect to local metabolic changes. This technique may also be experimentally applied in humans for measurements in adipose tissue. In the present invention, the release of serotonin in the mouse brain, in response to stimuli may be analyzed using this technique.

Microdialysis procedure involves the insertion through the guide cannula of a thin, needle-like perfusable probe (CMA/12.3 mm×0.5 mm) to a depth of 3 mm in striatum beyond the end of the guide. The probe is connected beforehand with tubing to a microinjection pump (CMA-/100). The probe may be perfused at 2 μl/min with Ringer's buffer (NaCl 147 mM; KCl 3.0 mM; CaCl₂ 1.2 mM; MgCl₂ 1.0 mM) containing 5.5 mM glucose, 0.2 mM L-ascorbate, and 1 μM neostigmine bromide at pH 7.4). To achieve stable baseline readings, microdialysis may be allowed to proceed for 90 minutes prior to the collection of fractions. Fractions (20 μl) may be obtained at 10 minute intervals over a 3 hour period using a refrigerated collector (CMA170 or 200). Baseline fractions may be collected, following the drug or combination of drugs to be tested, been administered to the animal. Upon completion of the collection, each mouse may be autopsied to determine accuracy of probe placement.

2. Behavioral Testing

Behavioral tests may be conducted as a follow on the transporter screens described above to further assess the efficacy of an given drug on a transporter variant. Such tests may include but are not limited to elevated plus-maze test, chronic mild stress test, forced swimming test, social defeat stress-induced anxiety test, or the light/dark test.

a. Elevated Plus-Maze Test in Mice

The apparatus may be based on that described by Pellow et al. (1985). In this procedure, the apparatus is elevated and contains two open and two enclosed arms, arranged so that the arms of the same type are opposite to each other. The apparatus is equipped with infrared beams and sensors capable of measuring arm activity for a given period of time. In addition, mice may be observed via video link by an observer located in an adjacent room. This arrangement allowed the recording of attempts at entry into open arms followed by avoidance responses, including stretched attend posture (the mouse stretches forward and retracts to original position). Tests may be performed 60 min after p.o. administration of the drugs.

b. Light/Dark Test in Mice

For this test, the apparatus may be based on that described by Belzung et al. (1989). For example, the apparatus may consist of two poly(vinyl chloride) boxes (20×20×14 cm), one of which is darkened. A desk lamp may be placed 20 cm above the lit box provided the room illumination. An opaque plastic tunnel (5×7×10 cm) may be used to separated the dark box from the illuminated one. The apparatus may be equipped with infrared beams capable of recording during a specific time period: (i) time spent by mice in the lit box, and (ii) number of tunnel crossings. Tests may be performed 30 min after i.p. administration of the drugs.

C. Forced Swimming Test in Mice

The forced swim test (FST) is widely used in the art for screening substances with a potential antidepressant effect. This procedure was originally described by Porsolt et al. (1977) however, modification may be made. Basically, the duration of immobility of the mice is measured for a given time period. The immobility observed by the FST is interpreted as “behavioral despair.”

3. Transgenic Animals

A transgenic animal of the present invention may involve an animal in which an altered transporter molecule is expressed in cells of the animal. Alternatively, a wild-type transporter may be expressed temporally or spatially in a manner different than a non-transgenic animal, or in a different amount than the endogenously expressed version of the transgene. In addition, the invention contemplates creation of “knock-out” animals to eliminate endogenous transporter expression, as well as “knock-in” animal where an exogenous transporter replaces the endogenous transporter.

In a general aspect, a transgenic animal is produced by the integration of a given transgene into the genome in a manner that permits the expression of the transgene, or by disrupting the wild-type gene, leading to a knockout of the wild-type gene. Methods for producing transgenic animals are generally described by Wagner and Hoppe (U.S. Pat. No. 4,873,191; which is incorporated herein by reference; Brinster et al. 1985; which is incorporated herein by reference in its entirety; and in Hogan, 1994; which is incorporated herein by reference in its entirety).

U.S. Pat. No. 5,639,457 is also incorporated herein by reference to supplement the present teaching regarding transgenic pig and rabbit production. U.S. Pat. Nos. 5,175,384; 5,175,385; 5,530,179, 5,625,125, 5,612,486 and 5,565,186 are also each incorporated herein by reference to similarly supplement the present teaching regarding transgenic mouse and rat production. Transgenic animals may be crossed with other transgenic animals or knockout animals to evaluate phenotype based on compound alterations in the genome.

As used herein, the term “transgene” means an exogenous gene introduced into a mouse through human intervention, e.g., by microinjection into a fertilized egg or by other methods known to those of average skill in the art. The term includes copies of the exogenous gene present in descendants of the mouse into which the exogenous gene was originally introduced. Likewise, the term “transgenic mouse” includes the original mouse into which the exogenous gene was introduced, as well as descendants of the original mouse so long as such descendants carry the transgene.

The transgenic animal of the invention may be produced by introducing transgenes into the germline of the animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the serotonin transporter transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host.

Retroviral infection can also be used to introduce transgene into a non-human animal. The developing non-human embryo can be cultured in vitro to the blastocyst stage. During this time, the blastomeres can be targets for retroviral infection (Jaenich, 1976). Efficient infection of the blastomeres is obtained by enzymatic treatment to remove the zona pellucida (Manipulating the Mouse Embryo, 1986). The viral vector system used to introduce the transgene is typically a replication-defective retrovirus carrying the transgene (Jahner et al., 1985; Van der Putten et al., 1985). Transfection is easily and efficiently obtained by culturing the blastomeres on a monolayer of virus-producing cells (Van der Putten, et al., 1985; Stewart et al., 1987). Alternatively, infection can be performed at a later stage. Virus or virus-producing cells can be injected into the blastocoele (Jahner et al., 1982). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al., 1982).

Embryonal stem cells (ES) may also be used for introducing transgenes. ES cells are obtained from pre-implantation embryos cultured in vitro and fused with embryos (Evans et al., 1981; Bradley et al., 1984; Gossler et al., 1986; and Robertson et al., 1986). Transgenes can be efficiently introduced into the ES cells by DNA transfection or by retrovirus-mediated transduction. Such transformed ES cells can thereafter be combined with blastocysts from a animal. The ES cells thereafter colonize the embryo and contribute to the germ line of the resulting chimeric animal (Jaenisch, 1988). ES cell are oftent used to create “knock in” or “knock out” animals.

A DNA fragment may be introduced into a mouse genome to produce a transgenic line of mice. The DNA fragment, usually a linear portion of a plasmid designed to express a gene under known genetic control elements, is microinjected into a pronucleus of a blastocyst or zygote. The injected cell develops following its introduction into the oviduct of pseudo-pregnant recipient female mice. If the DNA integrates into one of the chromosomes (it usually does so during the first few cell divisions of preimplantation development), then the transgenic founder mice are mosaic for the presence of the injected DNA. Founders produced in this way are very likely to have germ cells with the integrated transgene, and therefore will be able to transmit the integrated gene. In this way, transgenic lines of mice are produced, in which all cells of a transgenic mouse contain the transgene. The number of copies of the integrated DNA fragment can vary from one to several hundred, primarily arranged in a head-to-tail array.

The number of copies of the transgene constructs which are added to the cell is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. There is often an advantage to having more than one functioning copy of each of the inserted exogenous DNA sequences to enhance the phenotypic expression of the exogenous DNA sequences. For the purposes of this invention a zygote is essentially the formation of a diploid cell which is capable of developing into a complete organism. Generally, the a will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the cell or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the cell. The biological limit of the number and variety of DNA sequences will vary depending upon the particular cell and functions of the exogenous genetic material and will be readily apparent to one skilled in the art, because the genetic material, including the exogenous genetic material, of the resulting cell must be biologically capable of initiating and maintaining the differentiation and development of the zygote into a functional organism.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

V. Treatment of Disease States

A. Disease States

In other particular embodiments, the present invention provides a methods of treating a neurologic or psychiatric condition associated with transporter dysfunction comprising administering to a subject in need thereof a therapeutically effective amount of ubiquitination modulator (agonist or antagonist). Neurologic or psychiatric conditions that may be treated in this fashion include, but are not limited to, obsessive compulsive disorders (OCDs), autism, generalized anxiety disorders, pathological aggression, schizophrenia, schizotypal personality disorder, psychosis, a schizoaffective disorder, manic type disorder, a bipolar affective disorder, a bipolar affective (mood) disorder with hypomania and major depression (BP-II), a unipolar affective disorder, unipolar major depressive disorder, dysthymic disorder, a phobia, a panic disorder, a somatization disorder, hypochondriasis, drug abuse, autonomic dysfunction, ADHD, or an attention deficit disorder.

B. Agents

In the context of the present invention, a number of different agents are envisioned that may prove useful in modulating the ubiquitination, and hence activity, of membrane-bound transporters. For example, it is envisioned that protein kinase C will play an important role in the modulation of ubquitination of SERT, NET and DAT. Thus, PKC, nucleic acids encoding PKC, and PKC modulators (inhibitors: isoquinolinesulfonamides, PKC inhibitor 20-28, PKC inhibitor 19-31, PKC inhibitor 19-36, PKC inhibitor EGF-R fragment 651-658, PKC inhibitor C2-4, Ro 318220 and GF 109203X; agonists: phorbol esters, diacylglycerol). Other modulators include ubiquitinating enzyme E1A and nucleic acids coding therefor, ubiquitin hydrolase and nucleic acids coding therefor, ubiquitin substrates and ubiquitin inhibitors (Aclacinomycin A, Streptomyces galilaeus, AdaAhx₃L₃VS, AdaLys(Bio)Ahx₃L₃VS, ALLM, ALLN, Proteasome Inhibitor VII, Antiprotealide, Epoxomicin, Synthetic, Lactacystin, Synthetic, clasto-Lactacystin b-Lactone, a-Methylomuralide, MG-115, MG-132, NLVS, NP-LLL-VS, Proteasome Inhibitors I, II, III, and IV, Ro106-9920, Tyropeptin A, Ubiquitin aldehyde, UCH-L1, UCH-L3, and YU101).

C. Pharmaceutical Formulations

The present invention also contemplates the administration of substance(s) as therapeutic agents for the treatment of transporter-related diseases. The substance(s) may be prepared in pharmaceutical compositions. Generally, this will entail preparing compositions that are essentially free of pyrogens, as well as other impurities that could be harmful to humans or animals.

One will generally desire to employ appropriate salts and buffers. Aqueous compositions of the present invention comprise an effective amount of the neurotransmitter transporter modulator dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Supplementary active ingredients also can be incorporated into the compositions.

Administration of these compositions according to the present invention will be via any common route so long as the target tissue is available via that route. This includes administration may be by systemic or parenteral methods including intravenous injection, intraspinal injection, intracerebral, intradermal, subcutaneous, intramuscular, intraperitoneal methods. Depending on the nature of the modulator administration may also be via oral, nasal, buccal, rectal, vaginal or topical. Such compositions would normally be administered as pharmaceutically acceptable compositions, described supra.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The composition may be formulated as a “unit dose.” For example, one unit dose could be dissolved in 1 ml of isotonic NaCl solution and either added to 1000 ml of hypodermoclysis fluid or injected at the proposed site of infusion, (see for example, “Remington's Pharmaceutical Sciences,” 15^{th} Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Ubiquitination of NET in Transfected CAD Cells.

Immunoprecipitation of ubiquitinated NET. cDNAs of NET and HA-Ub were transfected into CAD cells 2 days prior to immunoprecipitation (IP). Left: The complexes were retrieved either by IP with anti-HA for Ub, and probed with anti-NET. Right: The complexes were retrieved by IP with anti-NET, and immunoblotted with anti-HA for Ub. N (NET only), N+U (NET and Ub co-transfection), U (Ub only). Increase of the sizes of NET by ubiquitination. CAD cells were transfected with NET and HA-Ub, immunoprecipitated with anti-HA for Ub, then probed with anti-NET. Note the increase of sizes in immunoprecipitated NET (NET:Ub complexes), compared to NET in total. Right panel: Size of proteins were measured form the left panel of western blot. Standard curve was prepared using migration pattern of molecular weight (MW) marker to determine sizes. Both 90 kDa and 60 kDa bands were shifted to higher MW forms by 7 kDa. Size of high MW bands at the top of gel was not determined.

Ubiquitination of DAT (Dopamine Transporters) and SERT (Serotonin Transporters) in Transfected CAD Cells.

cDNAs for HA-ubiquitin (Ub) and DAT or SERT were cotransfected into CAD cells 2 days prior to immunoprecipitation (IP). The complexes of Ub: transporters were immunoprecipitated by anti-HA and probed with anti-DAT or anti-SERT. Ubiquitinated DAT and SERT (IP) migrated at larger sizes compared to DAT or SERT in total.

Ubiquitination of DAT in Striatum.

Striatum was dissected from mouse brains in homogenized in 10 mM HEPES, 300 mM sucrose, pH 7.4. Crude synaptosomes were -obtained by a sequential centrifugation of a 5 min at 1000 ×g, followed by a 20 min at 16,000 ×g. Proteins extracted from the synaptosomes by an one hour incubation in 10 mM HEPES, 300 mM NaCl, 1% TRITON X 100 and protease inhibitors at cold. The lysates were pre-cleared with protein A and G beads. Aliquots of the pre-cleared lysates were incubated with either mouse IgG or monoclonal anti-DAT. Antibodies captured by protein A and G beads were subjected to 10% SDS-PAGE, followed by western blot with anti-Ub. The membranes were stripped and re-probed with anti-DAT.

Acute Regulation of NET Ubiquitination.

CAD cells were transfected with NET and HA-Ub, and incubated at 37° C. IP was carried out using anti-HA (IP for Ub), followed by immunoblot with anti-NET. Prior to IP, cells were pre-incubated in media containing either vehicle (control), 1 mM PMA, or 1 mM methacholine (meth) for 30 min. PMA and methacholine increased ubiquitination of NET.

Chronic Regulation of NET Ubiquitination by Anti-Depressants.

CAD cells were transfected with NET and HA-Ub. After 24 hours, desipramine (Dmi) was added to the cells. CAD cells were incubated for additional 3 days prior to IP. IP was carried out using anti-HA (IP for Ub), followed by immunoblot with anti-NET. Desipramine reduced ubiquitination of NET (IP) and increased NET proteins (total). The “total” membrane were stained with Ponceau-S, showing no change in the amounts of other cellular proteins by desipramine treatment.

Proteasome Influence on NET Transport and NET Proteins.

Transport Assay. CAD-his-hNET cells in 24 well plates were incubated in MG132 (10 mM) for 0, 1, 4 and 24 hrs prior to NE transport assay in triplicates. Protein Assay. CAD cells transfected with NET and Ub were preincubated with MG132 for 4 hrs prior to cell lysis and immunoblot with anti-NET for protein analysis.

### Example 2

### Results

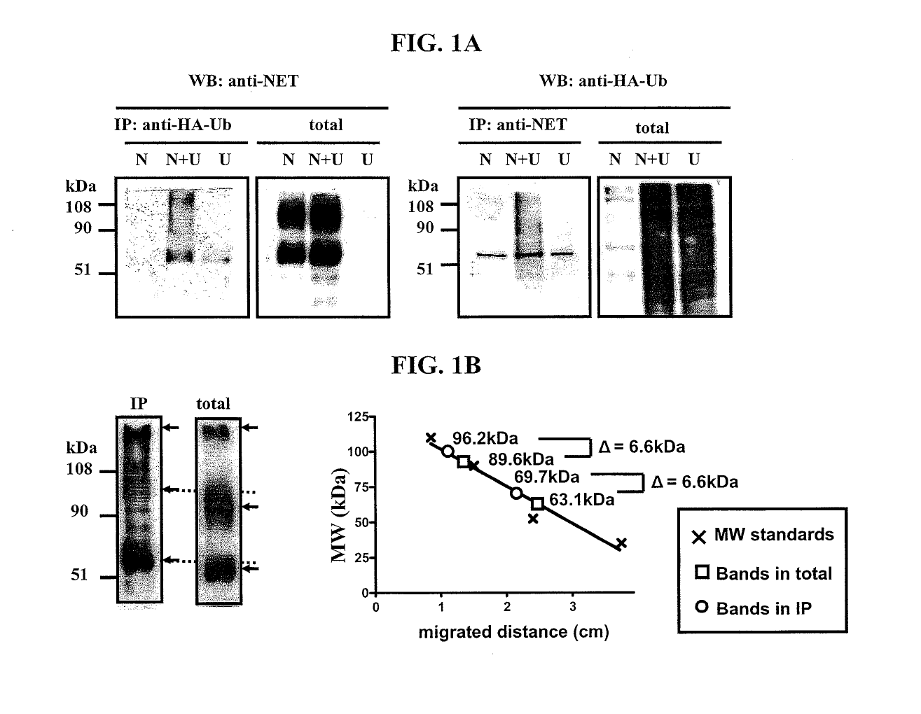

In order to assess ubiquitination of NET, immunoprecipitation studies were performed. cDNAs of NET and HA-Ub were transfected into CAD cells and complexes were retrieved either by IP with anti-HA for Ub, and probed with anti-NET, or vice versa. As can be seen, in both cases, a band from the NET, Ub and NET+Ub comigrated, indicating ubiquitination of NET (FIG. 1A). Using CAD cells transfected with NET and HA-Ub, followed by immunoprecipitating with anti-HA for Ub and probing with anti-NET, a stepwise increase in the size of in immunoprecipitated NET was shown. Measured size of NET, plotted on a standard curve, revealed that both 90 kDa and 60 kDa bands were shifted to higher MW forms by approx. 7 kDa (FIG. 1B, right panel).

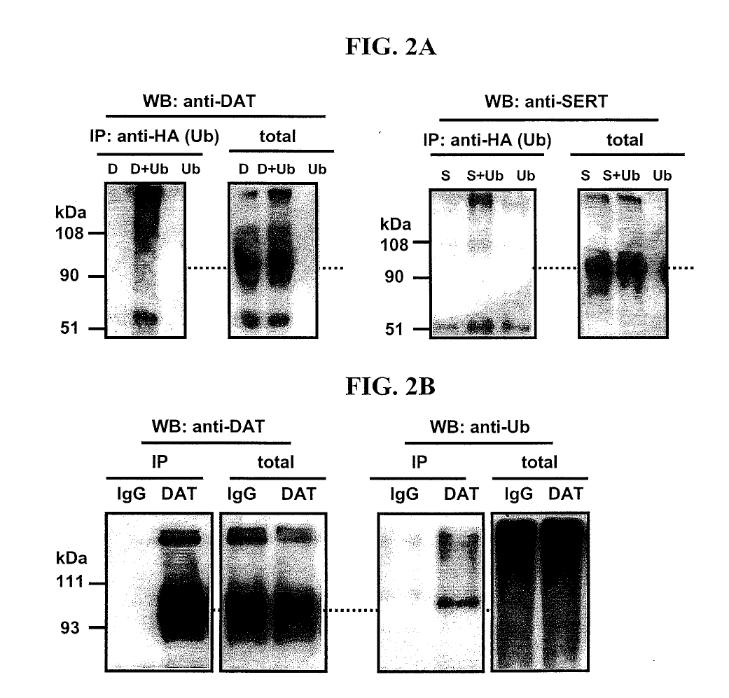

Next, ubiquitination of DAT (dopamine transporters) and SERT (serotonin transporters) was assessed in transfected CAD cells, using an approach similar to that outlined above for NET. As shown in FIG. 2A, SERT/DAT, Ub and SERT+Ub/DAT+Ub comigrated, indicating ubiquitination of these transporters. Further, ubiquitination of DAT in striatum was assessed by IP-Westem blot, as described above. Ubiquitination in these tissues was observed (FIG. 2B).

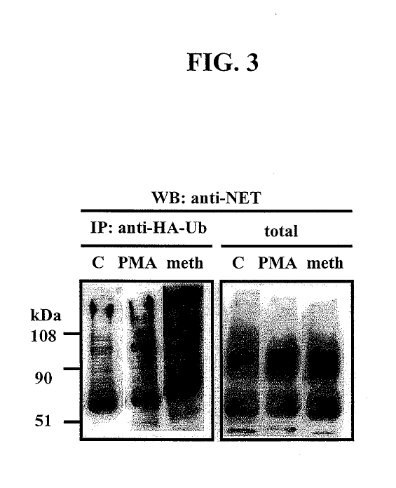

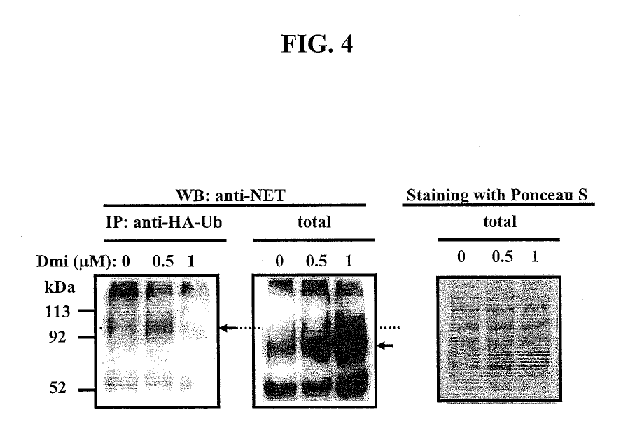

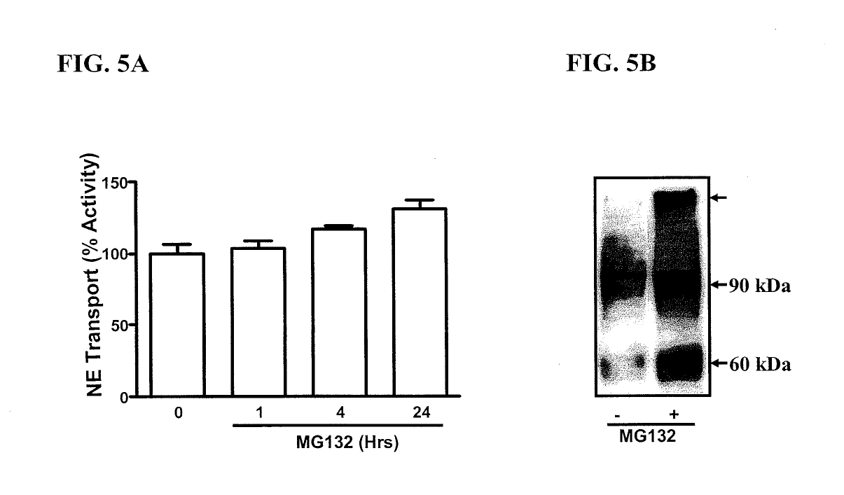

Acute regulation of NET ubiquitination by PMA and methacholine (meth) was assessed. CAD cells were transfected with NET and HA-Ub, and incubated at 37° C. IP was carried out using anti-HA (IP for Ub), followed by immunoblot with anti-NET. One mM PMA and 1 mM meth both increased ubiquitination of NET (FIG. 3). Chronic regulation of ubiquitination of NET was assessed in CAD cells transfected with NET and HA-Ub. After 24 hours, desipramine (Dmi) was added to the cells. Desipramine reduced ubiquitination of NET and increased total NET proteins (FIG. 4). It was also determined that inhibition of proteasomes with MG132 (10 mM) increase NET transport and NET protein concentration in CAD cells transfected with NET and Ub constructs. FIGS. 5A-5B.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,684,611
U.S. Pat. No. 4,873,191
U.S. Pat. No. 4,952,500
U.S. Pat. No. 5,175,384
U.S. Pat. No. 5,175,385
U.S. Pat. No. 5,302,523
U.S. Pat. No. 5,312,734
U.S. Pat. No. 5,384,253
U.S. Pat. No. 5,418,162
U.S. Pat. No. 5,424,185
U.S. Pat. No. 5,464,765
U.S. Pat. No. 5,530,179
U.S. Pat. No. 5,565,186
U.S. Pat. No. 5,580,859
U.S. Pat. No. 5,589,466
U.S. Pat. No. 5,612,486
U.S. Pat. No. 5,625,125
U.S. Pat. No. 5,639,457
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,925,565
U.S. Pat. No. 5,935,819
U.S. Pat. No. 5,945,100
U.S. Pat. No. 5,981,274
U.S. Pat. No. 5,994,624
U.S. Pat. No. 5,838,002
U.S. Pat. No. 5,788,166
U.S. Pat. No. 5,757,994
RE 35,413
U.S. Pat. No. 6,756,586
U.S. Pat. No. 5,572,023
U.S. Pat. No. 5,986,258
Abriel et al., J. Clin. Invest., 103:667-673, 1999.
Apparsundaram et al., J. Pharmacol. Exp. Ther., 287:733-743, 1998a.
Apparsundaram et al., J. Pharmacol. Exp. Ther., 287:744-751, 1998b.
Apparsundaram et al., J. Pharmacol. Exp. Ther., 299:666-677, 2001.
Arnsten, Adv. Neurol., 85:333-341, 2001.
Ausubel et al., In: Current Protocols in Molecular Biology, John, Wiley & Sons, Inc, New York, 1994.
Bahr et al., J. Mass Spectrom., 32:1111-1116, 1997.
Baichwal and Sugden, In: Gene Transfer, Kucherlapati (Ed.), NY, Plenum Press, 117-148, 1986.
Bauman et al., J. Neurosci., 20:7571-7578, 2000.
Belzung et al., Psychopharmacology (Berl). 97(3):388-91, 1989.
Bentzley et al., Anal Chem., 68(13):2141-2146, 1996.
Blackman et al., Biophys. J., 71(1):194-208, 1996.
Blakely et al., Anal. Biochem. 194:302-308, 1991.
Bradley and Blakely, J. Neurochem., 69:1356-1367, 1997.
Bradley et al., Nature, 309:255-258, 1984.
Brinster et al., Proc. Natl. Acad. Sci. USA, 82(13):4438-4442, 1985.
Bucknall et al., J. Am. Soc. Mass Spectrom., 13(9):1015-1027, 2002.
Burbea et al., Neuron., 35:107-120, 2002.
Caprioli et al., Anal. Chem., 69:4751, 1997.
Carbonelli et al., FEMS Microbiol. Lett., 177(1):75-82, 1999.
Carneiro et al., J. Neurosci., 22:7045-7034, 2002.
Carneiro et al., J. Neuroscience, 22:7045-7054, 2002.
Chandler et al., Proc. Natl. Acad. Sci. USA, 94(8):3596-601, 1997.
Chaurand et al., Anal. Chem., 71(23):5263-526370, 1999.
Chen and Okayama, Mol. Cell Biol., 7(8):2745-2752, 1987.
Chen et al., J. Biol. Chem., 273:12675-12681, 1998.
Chen et al., Nat. Biotechnol., 19:537-542, 2001.
Ciechanover and Brundin, Neuron, 40:427-446, 2003.
Cocea, Biotechniques, 23(5):814-816, 1997.
Colledge et al., Neuron., 40:595-607, 2003.
Cook and Leventhal, Curr. Opin. Pediatrics, 8:348-354, 1996.
Coupar et al., Gene, 68:1-10, 1988.
Delgado and Moreno, J. Clin. Psychiatry, 61:5-12, 2000.
Delgado, J. Clin. Psychiatry, 61:7-11, 2000.
Desiderio et al., J. Mass Spectrom., 35(6):725-733, 2000.
Desiderio et al., Methods Mol. Biol., 61:57-65, 1996.
Duncan et al., Rapid Commun. Mass Spectrom., 7(12):1090-1094, 1993.
Edwards, Ann. Neurol., 34(5):638-645, 1993.
Ehlers, Nat. Neurosci., 6:231-242, 2003.
Eng et al., J. Amer. Soc. Mass Spectrometry, 5:976-989, 1994.
Evans et al., Nature, 292:154-156, 1981.
Faulstich et al., Anal. Chem., 69(21):4349-4353, 1997.
Fechheimer, et al., Proc Natl. Acad. Sci. USA, 84:8463-8467, 1987.
Fenn et al., Science, 246(4926):64-71, 1989.
Fozzard, In: Peripheral actions of 5-hydroxytryptamine, Oxford University Press, New York, 1989.
Fraley et al., Proc. Natl. Acad. Sci. USA, 76:3348-3352, 1979.
Friedmann, Science, 244:1275-1281, 1989.
Galli et al., Proc. Natl. Acad. Sci. USA, 95(22):13260-13265, 1998.
Gershon, Aliment Pharmacol. Ther., 13(2):15-30, 1999.
Giros et al., Nature, 379(6566):606-612, 1996.
Gobom et al., Anal. Chem., 72(14):3320-3326, 2000.
Gopal, Mol. Cell Biol., 5:1188-1190, 1985.
Gossler et al., Proc. Natl. Acad. Sci. USA 83: 9065-9069, 1986.
Graham and Van Der Eb, Virology, 52:456-467, 1973.
Gross-Isseroff et al., Archives of General Psychiatry, 46:237-241, 1989.
Gu et al., J. Biol. Chem., 269:7124-7130, 1994.
Gygi et al., Nat. Biotechnol., 17:994-999, 1999.
Harland and Weintraub, J. Cell Biol., 101(3):1094-1099, 1985.
Hermonat and Muzycska, Proc. Natl. Acad. Sci. USA, 81:6466-6470, 1984.
Hoffman et al., Science, 254:579-580, 1991.
Hogan et al., In: Manipulating the Mouse Embryo: A Laboratory Manual, 2nd ed., Cold Spring Harbor Laboratory Press, 1994.
Horak et al., Rapid Commun. Mass Spectrom., 15(4):241-248, 2001.
Horwich et al. J. Virol., 64:642-650, 1990.
Insel et al., Annals New York Academy of Sciences, 574-586, 1990.
Jacobs and Azmitia, Physiological Rev., 72:165-229, 1992.
Jaenich, Proc. Natl. Acad. Sci. USA, 73(4):1260-1264, 1976.
Jaenisch, Science 240:1468-1474, 1988.
Jahner et al., Nature 298:623-628, 1982.
Jahner et al., Proc. Natl. Acad. Sci. USA, 82:6927-6931, 1985.
Jayanthi et al., J. Biol. Chem., 279:19315-19326, 2004.
Jespersen et al., Anal. Chem., 71(3):660-666, 1999.
Jiang et al., J. Agric. Food Chem., 48:3305, 2000.
Jiang et al., J. Biol. Chem., 279(52):54380-54386, 2004.
Jiang et al., Neuron., 21:799-811, 1998.
Kabarle et al., Anal. Chem. 65(20):972A-986A, 1993.
Kaeppler et al., Plant Cell Reports, 9:415-418, 1990.
Kanazawa et al., Biol. Pharm. Bull., 22(4):339-346, 1999.
Kaneda et al., Science, 243:375-378, 1989.
Kato et al, J. Biol. Chem., 266:3361-3364, 1991.
Kazmaier et al., Anesthesiology, 89(4):831-817, 1998.
Kilic & Rudnick, Proc. Nat'l Acad. Sci. USA 97:3106-3111, 2000.
Klimek et al., J. Neurosci., 17(21):8451-8458, 1997.
Kocabas et al., J. Neurochem. 85(6):1513, 2003.
Levenson et al., Hum. Gene Ther., 9(8):1233-1236, 1998.
Li et al., Proc. Natl. Acad. Sci. USA, 97(4):1566-1571, 2000.
Lovelace et al., J. Chromatogr., 562(1-2):573-584, 1991.
Lynn et al., J. Mol. Evol., 48(5):605-614, 1999.
Maniatis, et al., Molecular Cloning, A Laboratory Manual, Cold Spring Harbor Press, Cold Spring Harbor, N.Y., 1990.
Manipulating the Mouse Embryo, Hogan eds. (Cold Spring Harbor Laboratory Press, Cold Spring Harbor, 1986
Marie et al., Anal. Chem., 72(20):5106-5114, 2000.
Mason and Liebler, J. Proteome. Res., 2(3):265-272, 2003.
Meltzer, Annals New York Academy of Sciences, 486-499, 1990.
Miketova and Schram, Mol. Biotechnol., 8(3):249-253, 1997.
Miner et al., J. Comp. Neurol., 427:220-234, 2000.
Mirgorodskaya et al., Rapid Commun. Mass Spectrom., 14(14):1226-1232, 2000.
Muddiman et al., Fres. J. Anal. Chem., 354:103, 1996.
Myat et al., Neuron., 35:447-459, 2003.
Nelson et al., Anal. Chem., 66:1408, 1994.
Nguyen et al., J. Chromatogr. A., 705(1):21-45, 1995.
Nicolas and Rubinstein, In: Vectors. A survey of molecular cloning vectors and their uses, Rodriguez and Denhardt, eds., Stoneham: Butterworth, 494-513, 1988.
Nicolau and Sene, Biochim. Biophys. Acta, 721:185-190, 1982.
Nicolau et al., Methods Enzymol., 149:157-176, 1987.
Omirulleh et al., Plant Mol. Biol., 21(3):415-28, 1993.
Ozsarac et al., J. Neurochem., 82:336-44, 2002.
Pak and Sheng, Science, 302:1368-1373, 2003.
Pelletier and Sonenberg, Nature, 334(6180):320-325, 1988.
Pellow et al., J. Pharm. Pharmacol., 37(8):560-563, 1985.
Porsolt et al., Nature, 266(5604):730-732, 1977.
Potrykus et al., Mol. Gen. Genet., 199(2):169-177, 1985.
Potter et al., Proc. Natl. Acad. Sci. USA, 81:7161-7165, 1984.
Ramamoorthy et al., Proc. Natl. Acad. Sci. USA, 90:2542-2546, 1993.
Rehman and Masson, Age Ageing, 30:279-287, 2001.
Remington's Pharmaceutical Sciences, 15^{th} ed., pages 1035-1038 and 1570-1580, Mack Publishing Company, Easton, Pa., 1980.
Ridgeway, In: Vectors. A survey of molecular cloning vectors and their uses, Stoneham: Butterworth, pp. 467-492, 1988.
Rippe, et al., Mol. Cell Biol., 10:689-695, 1990.
Robertson et al., Nature 322:445-448, 1986
Roepstorff, EXS., 88:81-97, 2000.
Sambrook et al., In: Molecular cloning, Cold Spring Harbor Laboratory Press, Cold Spring Harbor, N.Y., 2001.
Shenoy et al., Science, 294:1307-1313, 2001.
Staub et al., Embo. J., 16:6325-6336, 1997.
Stewart et al., EMBO J. 6:383-388, 1987.
Stoeckli et al., Nat. Med., 7(4):493-496, 2001.
Sung et al., In: Ubiquitination mechanisms in support of norepinephrine tansporter regulation, Society for Neuroscience, Washington, D.C., Prog. No 280, Oct. 6, 2004a (abstract).
Sung et al., In: Ubiquitination mechanisms in support of norepinephrine tansporter regulation, Society for Neuroscience, Washington, D.C., Prog. No 280, Oct. 24, 2004b (poster).
Sung et al., J. Neurosci., 23:1697-1709, 2003.
Takach et al., J. Protein Chem., 16:363, 1997.
Temin, In: Gene Transfer, Kucherlapati (Ed.), N.Y., Plenum Press, 149-188, 1986.
Torres et al., Neuron., 30:121-134, 2001.
Tur-Kaspa et al., Mol. Cell Biol., 6:716-718, 1986.
Van der Putten et al., Proc. Natl. Acad. Sci. USA 82:6148-6152, 1985.
Vaughan et al., J. Neurosci Methods, 40(1):9-16, 1991.
Villanueva et al., Enzyme Microb. Technol., 29:99, 1999.
Wada et al., Nucleic Acids Res. 18:2367-2411, 1990.
Wang et al., Anal. Chem., 72(21):5285-5289, 2000.
Wang et al., J. Agric. Food. Chem., 47:1549, 1999.
Wang et al., J. Agric. Food. Chem., 48:2807, 2000.
Wittmann et al., Biotechnol. Bioeng., 72:642, 2001.
Wong et al., Gene, 10:87-94, 1980.
Wu and Wu, Biochemistry, 27:887-892, 1988.
Wu and Wu, J. Biol. Chem., 262:4429-4432, 1987.
Wu et al., Anal. Chem., 70:456A, 1998.
Wu et al., Biochim. Biophys. Acta, 1466:315-327, 2000.
Yang et al., J. Agric. Food. Chem., 48:3990, 2000.
Zaluzec et al., Protein Expr. Purif, 6:109-123, 1995.
Zaro et al., Arch. Neurol., 60:337-341, 2003.
Zhong et al., Clin. Chem. ACTA., 313:147, 2001.
Zhou et al., Nat. Biotechnol., 20:512-515, 2002.
Zweigenbaum et al., Anal. Chem., 71(13):2294-300, 1999.
Zweigenbaum et al., J. Pharm. Biomed. Anal., 23(4):723-733, 2000.

What is claimed is:

1. A method of screening for agents that affect transporter function comprising:
(a) providing a membrane-bound transporter;
(b) contacting said membrane-bound transporter with a candidate substance;
(c) determining the ubiquitination of said transporter; and
(d) comparing the ubiquitination of said transporter in step (c) with the ubiquitination of said transporter in the absence of said candidate substance,
wherein a candidate substance that alters the ubiquitination of said transporter is an agent that affects transporter function.

2. The method of claim 1, wherein said transporter is a norephinephrine transporter, a serotonin transporter or a dopamine transporter.

3. The method of claim 1, wherein said transporter is located in an intact cell.

4. The method of claim 3, wherein said cell is a neuronal cell.

5. The method of claim 3, wherein said cell is recombinantly engineered to express said transporter.

6. The method of claim 3, wherein said cell is from a post-mortem tissue.

7. The method of claim 3, wherein said cell is from a tissue biopsy.

8. The method claim 1, wherein said transporter is located in a membrane fragment.

9. The method of claim 8, wherein said transporter was produced by cell-free translation.

10. The method of claim 1, wherein determining ubiquitination comprises an immunoassay with a ubiquitin-binding antibody.

11. The method of claim 1, wherein determining ubiquitination comprises mass spectrometry.

12. The method of claim 1, wherein labeled ubiquitin is provided exogenously to said cell.

13. The method of claim 1, further comprising measuring the ubiquitination of said transporter before and after contacting said transporter with said candidate substance.

14. The method of claim 1, wherein said candidate substance is a peptide, polypeptide, nucleic acid, lipid, carbohydrate, or organopharmaceutical drug.

15. The method of claim 14, wherein said candidate substance is a polypeptide or a nucleic acid coding therefor, wherein said polypeptide an enzyme.

16. The method of claim 15, wherein said enzyme is a protein kinase C.

17. The method of claim 14, wherein said candidate substance is an organopharmaceutical drug that modulates protein kinase C.

18. The method of claim 14, wherein said polypeptide is ubiquitin-activating enzyme E1A.

19. The method of claim 1, wherein said candidate substance is a ubiquitin substrate, a ubiquitin inhibitor or a ubiquitin hydrolase.

20. A method of modulating neuronal transporter function in a subject comprising administering to said subject a modulator of transporter ubiquitination.

21. The method of claim 20, wherein said transporter is a norephinephrine transporter, a serotonin transporter or a dopamine transporter.

22. The method of claim 20, wherein said subject is a human.

23. The method of claim 20, wherein said human suffers from mental illness, cardiovascular disease, autonomic dysfunction, ADHD or drug abuse.

24. The method of claim 20, wherein said modulator is a peptide, polypeptide, nucleic acid, lipid, carbohydrate, or organopharmaceutical drug.

25. The method of claim 24, wherein said modulator is an enzyme or a nucleic acid encoding an expression construct for an enzyme.

26. The method of claim 25, wherein said modulator is a protein kinase C.

27. The method of claim 24, wherein said modulator is an organopharmaceutical drug that modulates protein kinase C.

28. The method of claim 24, wherein said polypeptide is ubiquitin-activating enzyme E1A.

29. The method of claim 20, wherein said modulator is a ubiquitin substrate, a ubiquitin inhibitor or a ubiquitin hydrolase.

30. A transgenic mouse encoding a mutant transporter gene, the product of which exhibits reduced or no ubiquitination.

31. The transgenic mouse of claim 30, wherein said mouse is homozygous for said mutant transporter gene.

32. The transgenic mouse of claim 30, wherein said mouse is heterozygous for said mutant transporter gene.
